SEQUENCE LISTING (18 sequences)

SEQ ID NO: 1 - protein, 240 aa, human
LKGDEGIQGLRGPSGVPGLPALSGVPGALGPQGFPGLKGDQGNPGRTTIGAAGLPGRDGLPGPPGPPGPPSPEFETETLHNKESGFPGLRGEQGPKGNLGLKGIKGDSGFCACDGGVPNTGPPGEPGPPGPWGLIGLPGLKGARGDRGSGGAQGPAGAPGLVGPLGPSGPKGKKGEPILSTIQGMPGDRGDSGSQGFRGVIGEPGKDGVPGLPGLPGLPGDGGQGFPGEKGLPGLPGEKG

SEQ ID NO: 2 - protein, 119 aa, human
GEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQA

SEQ ID NO: 3 - protein, 210 aa, human
AVGVWVGNKQLGHLPRDEAARYVPVLASLTAQGWLPQVSARVWGTEWSDYEERRASFRGSVRLDLAEPHMLVPVNLPPSGQHRLLPTGAAIQVTGEEKHLDALAPLLRPEGECWAYVSLHEMVEQTARTSRTVVEVRVDGSRVGQLTPKMSGELLPAIRHLAQGDVTTTARAIIKGNRIKSEVVLYVARAHELPDTWLGPAAPAAPAQMQ

SEQ ID NO: 4 - protein, 102 aa, human
FYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTT

SEQ ID NO: 5 - protein, 90 aa, human
LGFGSGHFRCDSSRWCHDNGVNYKIGEKWDRQGENGQMMSCTCLGNGKGEFKCDPHEATCYDDGKTYHVGEQWQKEYLGAICSCTCFGGQ

SEQ ID NO: 6 - protein, 119 aa, human; identical to SEQ ID NO: 2

SEQ ID NO: 7 - protein, 396 aa, human
LKGDEGIQGLRGPSGVPGLPALSGVPGALGPQGFPGLKGDQGNPGRTTIGAAGLPGRDGLPGPPGPPGPPSPEFETETLHNKESGFPGLRGEQGPKGNLGLKGIKGDSGFCACDGGVPNTGPPGEPGPPGPWGLIGLPGLKGARGDRGSGGAQGPAGAPGLVGPLGPSGPKGKKGEPILSTIQGMPGDRGDSGSQGFRGVIGEPGKDGVPGLPGLPGLPGDGGQGFPGEKGLPGLPGEKGGGTTCTGAAGGTTCTGAAGGTGAAGGTGGTTCTGAAGGTTCTGAAGGTGAAGGTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTT

SEQ ID NO: 8 - protein, 378 aa, human
HHHHHHAVGVWVGNKQLGHLPRDEAARYVPVLASLTAQGWLPQVSARVWGTEWSDYEERRASFRGSVRLDLAEPHMLVPVNLPPSGQHRLLPTGAAIQVTGEEKHLDALAPLLRPEGECWAYVSLHEMVEQTARTSRTVVEVRVDGSRVGQLTPKMSGELLPAIRHLAQGDVTTTARAIIKGNRIKSEVVLYVARAHELPDTWLGPAAPAAPAQMQGGTTCTGAAGGTTCTGAAGGTGAAGGTGGTTCTGAAGGTTCTGAAGGTGAAGGTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTHHHHHH

SEQ ID NO: 9 - protein, 366 aa, human
HHHHHHAVGVWVGNKQLGHLPRDEAARYVPVLASLTAQGWLPQVSARVWGTEWSDYEERRASFRGSVRLDLAEPHMLVPVNLPPSGQHRLLPTGAAIQVTGEEKHLDALAPLLRPEGECWAYVSLHEMVEQTARTSRTVVEVRVDGSRVGQLTPKMSGELLPAIRHLAQGDVTTTARAIIKGNRIKSEVVLYVARAHELPDTWLGPAAPAAPAQMQGGTTCTGAAGGTTCTGAAGGTGAAGGTGGTTCTGAAGGTTCTGAAGGTGAAGGTLGFGSGHFRCDSSRWCHDNGVNYKIGEKWDRQGENGQMMSCTCLGNGKGEFKCDPHEATCYDDGKTYHVGEQWQKEYLGAICSCTCFGGQHHHHHH

SEQ ID NO: 10 - DNA, 720 bp, human
ttaaagggagatgagggaathcagggattamgaggaccawsaggagtaccaggattaccagcattawsaggagtaccaggagcattaggaccacagggatttccaggattaaagggagatcagggaaatccaggamgaacaacaathggagcagcaggattaccaggamgagatggattaccaggaccaccaggaccaccaggaccaccawsaccagagtttgagacagagacattacataataaggagwsaggatttccaggattamgaggagagcagggaccaaagggaaatttaggattaaagggaathaagggagatwsaggattttgtgcatgtgatggaggagtaccaaatacaggaccaccaggagagccaggaccaccaggaccatggggattaathggattaccaggattaaagggagcamgaggagatmgaggawsaggaggagcacagggaccagcaggagcaccaggattagtaggaccattaggaccawsaggaccaaagggaaagaagggagagccaathttawsaacaathcagggaatgccaggagatmgaggagatwsaggawsacagggatttmgaggagtaathggagagccaggaaaggatggagtaccaggattaccaggattaccaggattaccaggagatggaggacagggatttccaggagagaagggattaccaggattaccaggagagaaggga

SEQ ID NO: 11 - DNA, 306 bp, human
ttttattcatgtacaacagagggamgacaggatggacatttatggtgttcaacaacatcaaattatgagcaggatcagaagtattcattttgtacagatcatacagtattagtacagacamgaggaggaaattcaaatggagcattatgtcattttccatttttatataataatcataattatacagattgtacatcagagggamgamgagataatatgaagtggtgtggaacaacacagaattatgatgcagatcagaagtttggattttgtccaatggcagcacatgaggagathtgtacaaca

SEQ ID NO: 12 - DNA, 1149 bp, human
ctcgagaaaagagaggctgaagctcatcatcatcatcatcatttaaagggagatgagggaattcagggattacgaggaccatgaggagtaccaggattaccagcattatgaggagtaccaggagcattaggaccacagggatttccaggattaaagggagatcagggaaatccaggacgaacaacaattggagcagcaggattaccaggacgagatggattaccaggaccaccaggaccaccaggaccaccatgaccagagtttgagacagagacattacataataaggagtgaggatttccaggattacgaggagagcagggaccaaagggaaatttaggattaaagggaattaagggagattgaggattttgtgcatgtgatggaggagtaccaaatacaggaccaccaggagagccaggaccaccaggaccatggggattaattggattaccaggattaaagggagcacgaggagatcgaggatgaggaggagcacagggaccagcaggagcaccaggattagtaggaccattaggaccatgaggaccaaagggaaagaagggagagccaattttatgaacaattcagggaatgccaggagatcgaggagattgaggatgacagggatttcgaggagtaattggagagccaggaaaggatggagtaccaggattaccaggattaccaggattaccaggagatggaggacagggatttccaggagagaagggattaccaggattaccaggagagaagggaggttctgaaggttctgaaggtgaaggtggttctgaaggttctgaaggtgaaggtttttattcatgtacaacagagggamgacaggatggacatttatggtgttcaacaacatcaaattatgagcaggatcagaagtattcattttgtacagatcatacagtattagtacagacamgaggaggaaattcaaatggagcattatgtcattttccatttttatataataatcataattatacagattgtacatcagagggamgamgagataatatgaagtggtgtggaacaacacagaattatgatgcagatcagaagtttggattttgtccaatggcagcacatgaggagathtgtacaacacatcatcatcatcatcattaatctaga

SEQ ID NO: 13 - DNA, 15 bp, artificial sequence
  rhCol-rhFN-F
ctcgagaaaagagag

SEQ ID NO: 14 - DNA, 15 bp, artificial sequence
  rhCol-rhFN-R
catcattaatctaga

SEQ ID NO: 15 - DNA, 18 bp, artificial sequence
  rhCol-F
gaattcttaaagggagat

SEQ ID NO: 16 - DNA, 17 bp, artificial sequence
  rhCol-R
tgtacaacagcggccgc

SEQ ID NO: 17 - DNA, 15 bp, artificial sequence
  rhFN-F
ctcgagttttattca

SEQ ID NO: 18 - DNA, 14 bp, artificial sequence
  rhFN- R
acaacagcggccgc

(19) United States
(12) Patent Application Publication
(10) Pub. No.: US 2023/0272046 A1
(43) Pub. Date: Aug. 31, 2023

(54) METHOD FOR PREPARING RECOMBINANT HUMAN EXTRACELLULAR MATRIX STRUCTURAL PROTEIN

(71) Applicants: Guangzhou Biopharmaceutical R&D Center of Jinan University Co., Ltd, Guangzhou, Guangdong (CN); GuangZhou JiDa Biological Technology Co., Ltd, Guangzhou, Guangdong (CN)

(72) Inventors: Qi XIANG, Guangzhou, Guangdong (CN); Yadong HUANG, Guangzhou, Guangdong (CN); Yating CHENG, Guangzhou, Guangdong (CN); Laiwu XUE, Guangzhou, Guangdong (CN)

(21) Appl. No.: 18/003,018

(22) PCT Filed: Dec. 4, 2020
(86) PCT No.: PCT/CN2020/133810

§ 371 Date: Dec. 22, 2022

(30) Foreign Application Priority Data
Jun. 24, 2020 (CN) 202010594000.4

(51) Int. Cl. C07K 14/78; C12N 15/62; C12N 15/81

(52) U.S. Cl. CPC C07K 14/78; C12N 15/62; C12N 15/815; C07K 2319/02

(57) ABSTRACT

The present invention falls within the field of genetic engineering. More particularly, disclosed is a gene for expressing a fusion protein of human collagen and human fibronectin, which is expressed and purified in vitro by means of a genetic engineering method. By means of using Pichia pastoris as an expression host cell, a new-type, highly active recombinant fusion protein can be provided.

### FIELD OF THE INVENTION

The invention belongs to the technical field of biological medicine, and in particular relates to a fusion protein.

### BACKGROUND OF THE INVENTION

Collagen is widely distributed in skins, bones, cartilage, teeth, tendons, blood vessels and ligaments of mammals. It is a group of protein families in human connective tissues, and it is also the most abundant protein in the body, accounting for about 25% to 33% of total protein. At present, collagen has become one of the most promising biological raw materials, which can be widely used in cosmetics, medical materials and other fields.

Fibronectin is a glycoprotein in extracellular matrix with high molecular weight (220-250 KD). It has multiple domains, and can bind to affinity site of cell surface receptor, collagen, fibrin and sulfated proteoglycan. Fibronectin is widely involved in the processes of cell migration, adhesion, proliferation, hemostasis and tissue repair, mobilizes the mononuclear phagocyte system to remove harmful substances in damaged tissues, and has a growth factor-like effect. As a matrix for cell culture, fibronectin can increase the adhesion rate and confluence rate of various cells, shorten the cell confluence period, make the cell shape and structure better, enhance the metabolic rate, and significantly increase the synthesis speed of DNA, RNA and protein.

### SUMMARY OF THE INVENTION

The present invention provides a fusion protein of recombinant human collagen-human fibronectin, which has significantly better effects on cell adhesion and migration than that of fibronectin alone, and is helpful for hemostasis and healing of wounds, and can promote the repair and regeneration of blood vessels and nerves. After mixed with appropriate adjuvant materials, the fusion protein can be further made into different dosage forms such as lyophilized powder and liposome, and added into cosmetics. The fusion protein can also be loaded in gel, sponge and other materials to prepare medical materials. The fusion protein of recombinant human collagen-human fibronectin in the present invention has the outstanding advantages of humanization, low immunogenicity and less prone to allergic reactions.

Therefore, the present provides the following aspects:
1. A fusion protein, which is obtained by fusion of human collagen and human fibronectin.
2. The fusion protein according to item 1, wherein the fusion protein comprises at least one domain of human type I collagen alpha chain and at least one domain of human type III fibronectin.
3. The fusion protein according to item 1 or 2, wherein the human collagen (rhCol) of the fusion protein is a short peptide formed by repeats of 10 to 50 amino acids, wherein the number of repeats is 10 to 20, and the amino acid sequence is SEQ ID NO.1, SEQ ID NO.2, or SEQ ID NO.3.
4. The fusion protein according to any one of items 1-3, wherein the human type III fibronectin (rhFN) of the fusion protein is one or more domains in 8-15 domains, and the amino acid sequence is SEQ ID NO.4, SEQ ID NO.5, or SEQ ID NO.6.
5. The fusion protein according to any one of items 1-4, wherein the N or C terminus of the human collagen is fused with the N or C terminus of the human fibronectin.
6. The fusion protein according to any one of items 1-5, wherein the human collagen is fused with the human fibronectin by a linker peptide; the linker peptide is preferably represented by (GGGS)n, wherein n is an integer of 1-5, preferably, the fusion protein further includes a Kex2 protease sequence, a secretion signal peptide sequence, a His tag for protein purification, or a combination thereof.
7. The fusion protein according to any one of items 1-6, wherein the amino acid sequence of the fusion protein is SEQ ID NO.7, SEQ ID NO.8, or SEQ ID NO.9.
8. A nucleic acid encoding the fusion protein according to any one of items 1-7.
9. An expression vector comprising the nucleic acid according to item 8, preferably the expression vector is selected from pPICZαA, pHIL, pPIC9k, and/or pPICZαB, more preferably pPICZαA or pHIL, most preferably pPICZαA.
10. A host cell comprising the expression vector according to item 9, preferably the host cell is a yeast cell, more preferably a Pichia cell, still more preferably GS115, X33 and KM71, most preferably GS115.

More specifically, the present invention provides a preparation method of recombinant human collagen-human fibronectin fusion protein, and uses thereof.
1. Specifically, the preparation method of human collagen-human fibronectin fusion protein of the present invention comprises the following steps: obtaining the DNA sequence encoding the fusion protein, constructing an appropriate recombinant expression vector to express the fusion protein in Pichia.
2. Specifically, the present invention also relates to the amino acid sequence of the fusion protein of human collagen-human fibronectin of the present invention, the nucleotide sequence encoding the fusion protein of human collagen-human fibronectin of the present invention, an expression vector expressing the fusion protein of human collagen-human fibronectin of the present invention, a host strain expressing the fusion protein of human collagen-human fibronectin of the present invention, and purification of the fusion protein of human collagen-human fibronectin of the present invention.
3. Specifically, the present invention provides a human collagen (rhCol), which comprises the amino acid sequence of SEQ ID NO.1, SEQ ID NO.2 or SEQ ID NO.3. The human collagen in the present invention is the functional domain of type I collagen a chain, and the truncated collagen can be expressed in large quantities in Pichia expression system, and has little effect on the activity, which solves the problem of difficulty in expressing full-length collagen and low expression level.
4. Specifically, the present invention provides a human fibronectin (rhFN), which comprises the amino acid sequence of SEQ ID NO.4, SEQ ID NO.5, or SEQ ID NO.6. The human fibronectin in the present invention is the functional domain of fibronectin type III. Full-length fibronectin is difficult to be secreted and expressed in Pichia cells. Said sequence in the present invention can not only be secreted and expressed in large quantities in yeast, but also has little effect on the activity, which solves the problem of difficulty in expressing full-length fibronectin and low expression level.
5. Specifically, the present invention provides a linker peptide. The general formula of the linker peptide is (GGGS)n, where n=an integer of 1 to 5, preferably n=3.
6. Specifically, the fusion protein sequence of the present invention also includes a Kex2 protease sequence, a secretion signal peptide sequence, a His tag for protein purification, or a combination thereof.
7. Specifically, the amino acid sequence of the fusion protein of the present invention is SEQ ID NO.7, SEQ ID NO.8, or SEQ ID NO.9.
8. Specifically, an expression vector for the fusion protein of the present invention includes the nucleic acid according to any one of the item 7, preferably the expression vector is pPICZαA, and the recombinant expression plasmid pPICZαA-rhCol-rhFN is constructed.
9. Specifically, for the fusion protein of the present invention, the expression strain is selected from GS115, X33 and KM71, preferably GS115.
10. The fusion protein according to claims 1-7 can be used as an active additive in the fields of tissue engineering, pharmacology or cosmetic skin care.

### BRIEF DESCRIPTION OF THE DRAWINGS

FIG. 1 shows the profiles of recombinant expression plasmids. A, pPICZαA-rhCol-rhFN; B, pPICZαA-rhCol; C, pPICZαA-rhFN.

FIG. 2 shows amplification pictures of the target fragment rhCol-rhFN (M is DL10000 DNA marker; lane 1 represents the target fragment rhCol-rhFN, about 1149 bp; lane 2 represents the target fragment rhFN, about 378 bp; lane 3 represents the target fragment rhCol, about 774 bp).

FIG. 3 shows the induction expression, purification and Western blot identification of proteins rhCol-rhFN, rhFN and rhCol (A, induction expression of rhCol-rhFN protein; B, induction expression of rhFN protein; C, induction expression of rhCol protein; Lane 1, uninduced samples; Lanes 2-4 represent samples induced for 24 h, 48 h, and 72 h respectively; D and d represent SDS-PAGE and Western blot of purified rhCol-rhFN protein respectively; E and e represent SDS-PAGE and Western blot of purified rhFN protein respectively; F and f represent SDS-PAGE and Western blot of purified rhCol protein respectively).

FIG. 4 shows the adhesion-promoting effect of the fusion protein rhCol-rhFN on cells (A, crystal violet staining to observe the cell morphology; B: the statistics of the number of cell attachment and extension. **, p<0.01 as compared with the control; ns, no significant difference).

FIG. 5 shows the schematic diagram of cell Wound-Healing Assay.

FIG. 6 shows the local lymph node test of fusion protein rhCol-rhFN (A, ATP content of lymphocytes in lymph nodes of mice. ###, p<0.001 as compared with control; *, p<0.05 as compared with rhCol-rhFN; B, Pinna weight of mouse. ####, p<0.0001 as compared to control; *, p<0.05 as compared to rhCol-rhFN).

### SEQUENCES INVOLVED IN THE PRESENT INVENTION

SEQ ID NO. 1
LKGDEGIQGLRGPSGVPGLPALSGVPGALGPQGFPGLKGDQGNPGRTTIGAAGLPGRDG
LPGPPGPPGPPSPEFETETLHNKESGFPGLRGEQGPKGNLGLKGIKGDSGFCACDGGVPN
TGPPGEPGPPGPWGLIGLPGLKGARGDRGSGGAQGPAGAPGLVGPLGPSGPKGKKGEPI
LSTIQGMPGDRGDSGSQGFRGVIGEPGKDGVPGLPGLPGLPGDGGQGFPGEKGLPGLPG
EKG
SEQ ID NO. 2
GEKWDRQGENGQMMSCTCLGNGKGEFKCDPHEATCYDDGKTYHVGEQWQKEYLGAI
CSCTCFGGQRGWRCDNCRRPGGEPSPEGTTGQSYNQYSQRYHQRTNTNVNCPIECFMP
LDVQA
SEQ ID NO. 3
AVGVWVGNKQLGHLPRDEAARYVPVLASLTAQGWLPQVSARVWGTEWSDYEERRAS
FRGSVRLDLAEPHMLVPVNLPPSGQHRLLPTGAAIQVTGEEKHLDALAPLLRPEGECWA
YVSLHEMVEQTARTSRTVVEVRVDGSRVGQLTPKMSGELLPAIRHLAQGDVTTTARAII
KGNRIKSEVVLYVARAHELPDTWLGPAAPAAPAQMQ
SEQ ID NO. 4
FYSCTTEGRQDGHLWCSTTSNYEQDQKYSFCTDHTVLVQTRGGNSNGALCHFPFLYNN
HNYTDCTSEGRRDNMKWCGTTQNYDADQKFGFCPMAAHEEICTT
SEQ ID NO. 5
LGFGSGHFRCDSSRWCHDNGVNYKIGEKWDRQGENGQMMSCTCLGNGKGEFKCDPH
EATCYDDGKTYHVGEQWQKEYLGAICSCTCFGGQ
SEQ ID NO. 6
GEKWDRQGENGQMMSCTCLGNGKGEFKCDPHEATCYDDGKTYHVGEQWQKEYLGAI
CSCTCFGGQRGWRCDNCRRPGGEPSPEGTTGQSYNQYSQRYHQRTNTNVNCPIECFMP
LDVQA
SEQ ID NO. 7
LKGDEGIQGLRGPSGVPGLPALSGVPGALGPQGFPGLKGDQGNPGRTTIGAAGLPGRDG
LPGPPGPPGPPSPEFETETLHNKESGFPGLRGEQGPKGNLGLKGIKGDSGFCACDGGVPN
TGPPGEPGPPGPWGLIGLPGLKGARGDRGSGGAQGPAGAPGLVGPLGPSGPKGKKGEPI
LSTIQGMPGDRGDSGSQGFRGVIGEPGKDGVPGLPGLPGLPGDGGQGFPGEKGLPGLPG
EKGGGTTCTGAAGGTTCTGAAGGTGAAGGTGGTTCTGAAGGTTCTGAAGGTGAAGG
TFYSCTTEGRQDGHLWCSTTSNYEQDQKYSFCTDHTVLVQTRGGNSNGALCHFPFLYN
NHNYTDCTSEGRRDNMKWCGTTQNYDADQKFGFCPMAAHEEICTT
SEQ ID NO. 8
HHHHHHAVGVWVGNKQLGHLPRDEAARYVPVLASLTAQGWLPQVSARVWGTEWSD
YEERRASFRGSVRLDLAEPHMLVPVNLPPSGQHRLLPTGAAIQVTGEEKHLDALAPLLR
PEGECWAYVSLHEMVEQTARTSRTVVEVRVDGSRVGQLTPKMSGELLPAIRHLAQGDV
TTTARALIKGNRIKSEVVLYVARAHELPDTWLGPAAPAAPAQMQGGTTCTGAAGGTTC
TGAAGGTGAAGGTGGTTCTGAAGGTTCTGAAGGTGAAGGTFYSCTTEGRQDGHLWCS
TTSNYEQDQKYSFCTDHTVLVQTRGGNSNGALCHFPFLYNNHNYTDCTSEGRRDNMK
WCGTTQNYDADQKFGFCPMAAHEEICTTHHHHHH
SEQ ID NO. 9
HHHHHHAVGVWVGNKQLGHLPRDEAARYVPVLASLTAQGWLPQVSARVWGTEWSD
YEERRASFRGSVRLDLAEPHMLVPVNLPPSGQHRLLPTGAAIQVTGEEKHLDALAPLLR
PEGECWAYVSLHEMVEQTARTSRTVVEVRVDGSRVGQLTPKMSGELLPAIRHLAQGDV
TTTARALIKGNRIKSEVVLYVARAHELPDTWLGPAAPAAPAQMQGGTTCTGAAGGTTC
TGAAGGTGAAGGTGGTTCTGAAGGTTCTGAAGGTGAAGGTLGFGSGHFRCDSSRWCH
DNGVNYKIGEKWDRQGENGQMMSCTCLGNGKGEFKCDPHEATCYDDGKTYHVGEQ
WQKEYLGAICSCTCFGGQHHHHHH
SEQ ID NO. 10
TTAAAGGGAGATGAGGGAATHCAGGGATTAMGAGGACCAWSAGGAGTACCAGGAT
TACCAGCATTAWSAGGAGTACCAGGAGCATTAGGACCACAGGGATTTCCAGGATTA
AAGGGAGATCAGGGAAATCCAGGAMGAACAACAATHGGAGCAGCAGGATTACCAG
GAMGAGATGGATTACCAGGACCACCAGGACCACCAGGACCACCAWSACCAGAGTT
TGAGACAGAGACATTACATAATAAGGAGWSAGGATTTCCAGGATTAMGAGGAGAG
CAGGGACCAAAGGGAAATTTAGGATTAAAGGGAATHAAGGGAGATWSAGGATTTT
GTGCATGTGATGGAGGAGTACCAAATACAGGACCACCAGGAGAGCCAGGACCACC
AGGACCATGGGGATTAATHGGATTACCAGGATTAAAGGGAGCAMGAGGAGATMGA
GGAWSAGGAGGAGCACAGGGACCAGCAGGAGCACCAGGATTAGTAGGACCATTAG
GACCAWSAGGACCAAAGGGAAAGAAGGGAGAGCCAATHTTAWSAACAATHCAGGG
AATGCCAGGAGATMGAGGAGATWSAGGAWSACAGGGATTTMGAGGAGTAATHGG
AGAGCCAGGAAAGGATGGAGTACCAGGATTACCAGGATTACCAGGATTACCAGGAG
ATGGAGGACAGGGATTTCCAGGAGAGAAGGGATTACCAGGATTACCAGGAGAGAA
GGGA
SEQ ID NO. 11
TTTTATTCATGTACAACAGAGGGAMGACAGGATGGACATTTATGGTGTTCAACAAC
ATCAAATTATGAGCAGGATCAGAAGTATTCATTTTGTACAGATCATACAGTATTAGT
ACAGACAMGAGGAGGAAATTCAAATGGAGCATTATGTCATTTTCCATTTTTATATAA
TAATCATAATTATACAGATTGTACATCAGAGGGAMGAMGAGATAATATGAAGTGGT
GTGGAACAACACAGAATTATGATGCAGATCAGAAGTTTGGATTTTGTCCAATGGCA
GCACATGAGGAGATHTGTACAACA
SEQ ID NO. 12
CTCGAGAAAAGAGAGGCTGAAGCTCATCATCATCATCATCATTTAAAGGGAGATGA
GGGAATTCAGGGATTACGAGGACCATGAGGAGTACCAGGATTACCAGCATTATGAG
GAGTACCAGGAGCATTAGGACCACAGGGATTTCCAGGATTAAAGGGAGATCAGGGA
AATCCAGGACGAACAACAATTGGAGCAGCAGGATTACCAGGACGAGATGGATTACC
AGGACCACCAGGACCACCAGGACCACCATGACCAGAGTTTGAGACAGAGACATTAC
ATAATAAGGAGTGAGGATTTCCAGGATTACGAGGAGAGCAGGGACCAAAGGGAAA
TTTAGGATTAAAGGGAATTAAGGGAGATTGAGGATTTTGTGCATGTGATGGAGGAG
TACCAAATACAGGACCACCAGGAGAGCCAGGACCACCAGGACCATGGGGATTAATT
GGATTACCAGGATTAAAGGGAGCACGAGGAGATCGAGGATGAGGAGGAGCACAGG
GACCAGCAGGAGCACCAGGATTAGTAGGACCATTAGGACCATGAGGACCAAAGGG
AAAGAAGGGAGAGCCAATTTTATGAACAATTCAGGGAATGCCAGGAGATCGAGGA
GATTGAGGATGACAGGGATTTCGAGGAGTAATTGGAGAGCCAGGAAAGGATGGAGT
ACCAGGATTACCAGGATTACCAGGATTACCAGGAGATGGAGGACAGGGATTTCCAG
GAGAGAAGGGATTACCAGGATTACCAGGAGAGAAGGGAGGTTCTGAAGGTTCTGAA
GGTGAAGGTGGTTCTGAAGGTTCTGAAGGTGAAGGTTTTTATTCATGTACAACAGAG
GGAMGACAGGATGGACATTTATGGTGTTCAACAACATCAAATTATGAGCAGGATCA
GAAGTATTCATTTTGTACAGATCATACAGTATTAGTACAGACAMGAGGAGGAAATT
CAAATGGAGCATTATGTCATTTTCCATTTTTATATAATAATCATAATTATACAGATTG
TACATCAGAGGGAMGAMGAGATAATATGAAGTGGTGTGGAACAACACAGAATTAT
GATGCAGATCAGAAGTTTGGATTTTGTCCAATGGCAGCACATGAGGAGATHTGTAC
AACACATCATCATCATCATCATTAATCTAGA
SEQ ID NO. 13
CTCGAGAAAAGAGAG
SEQ ID NO. 14
CATCATTAATCTAGA
SEQ ID NO. 15
GAATTCTTAAAGGGAGAT
SEQ ID NO. 16
TGTACAACAGCGGCCGC
SEQ ID NO. 17
CTCGAGTTTTATTCA
SEQ ID NO. 18
ACAACAGCGGCCGC

### DETAILED DESCRIPTION OF THE INVENTION

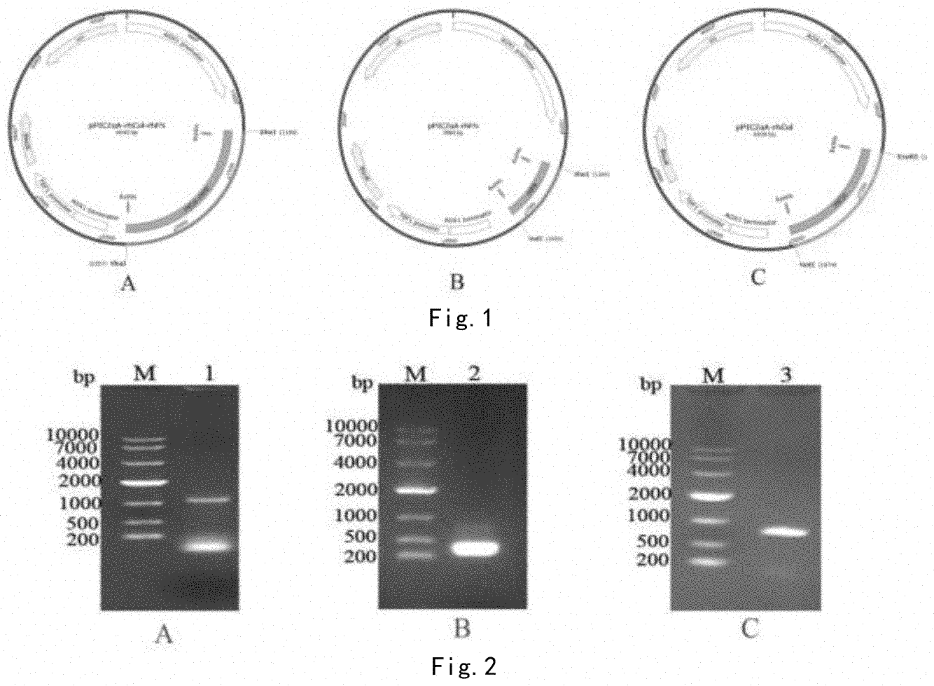

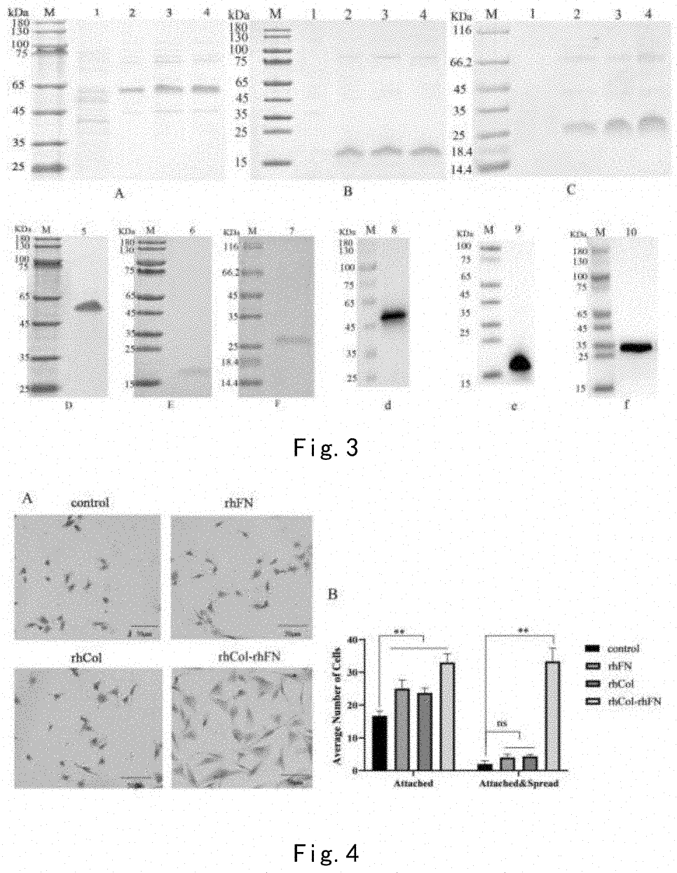

The invention provides a method for highly expressing a fusion protein rhCol-rhFN of human collagen and human fibronectin in Pichia pastoris, which comprises the following steps:
1. Construction of recombinant expression vector pPICZαA-rhCol-rhFN for the fusion protein: fragments of interest (human collagen (rhCol), human fibronectin (rhFN) and human collagen-human fibronectin (rhCol-rhFN)) were artificially synthesized (Shanghai Generay Biotechnology); wherein, the human collagen (rhCol) nucleotide sequence (SEQ ID NO. 10) was added with EcoR1 and Not1 restriction sites at both ends, the human fibronectin (rhFN) nucleotide sequence (SEQ ID NO. 11) was added with Xho1 and Not1 restriction sites at both ends, and the human collagen-human fibronectin fusion protein rhCol-rhFN nucleotide sequence (SEQ ID NO. 12) was added with restriction sites Xho1 and Xba1 at both ends; the expression plasmid pPICZαA (BioVector Science Lab, Inc.) was cleaved by EcoR1 and Not1, Xho1 and Not1, and Xho1 and Xba1, respectively, and then recovered; the corresponding target fragments and plasmids after digestion were ligated by T4 ligase to construct recombinant expression plasmids pPICZαA-rhCol, pPICZαA-rhFN and pPICZαA-rhCol-rhFN. FIG. 1 showed the plasmid profiles.
2. Transfection of Pichia pastoris GS115 strain by the recombinant plasmids: the recombinant plasmid pPICZαA-rhCol-rhFN (10 μg) linearized by Sal1 endonuclease, the recombinant plasmid pPICZαA-rhCol (10 μg) linearized by Sac1 endonuclease, and the recombinant plasmid pPICZαA-rhFN (10 μg) linearized by Sal1 endonuclease were respectively mixed with 80 μL Pichia pastoris GS115 (purchased from www.biofeng.com) competent cells, and the mixture was transferred to a 0.2 cm pre-cooled electroporation cup, subjected to an electric shock by 4-10 Milliseconds, added with 1 mL of ice-cooled 1 mol/L sorbitol solution and mixed well, inoculated on a YPD medium plate (containing 1% yeast extract, 2% peptone, 2% agar powder and Zeocin 100 μg/ml) by coating, inverted cultured at 30° C. for 2 days, until a single colony grown on the plate.
3. Screening of recombinant expression bacteria: the clones on the YPD plate were used as a template, added with primers rhCol-rhFN-F (SEQ ID NO. 13) and rhCol-rhFN-R (SEQ ID NO. 14), rhCol-F (SEQ ID NO. 15) and rhCol-R (SEQ ID NO. 16), as well as rhFN-F (SEQ ID NO. 17) and rhFN-R (SEQ ID NO. 18) (synthesized by BGI) (at 10 μM) by 0.5 μL respectively, then added with Premix Taq™ (TaKaRa Taq™ Version 2.0 plus dye) premixed enzyme, added with water to a total volume of 20 μL, and then subjected to the amplification of fragments of interest under PCR conditions (denaturation at 95° C. for 3 minutes, and then denaturation at 95° C. for 60 seconds, annealing at 60° C. for 30 seconds, extension at 72° C. for 120 seconds, for 30 cycles, and then extension at 72° C. for 10 minutes). The sizes of fragments of interest were detected by nucleic acid electrophoresis. As a result, as shown in FIG. 2, the fragments of interest were obtained.
4. Expression of fusion protein rhCol-rhFN, recombinant protein rhCol, and recombinant protein rhFN: the screened positive transformed strains were inoculated in 10 mL of YPD medium, cultured at 230 rpm, 30° C. for 18-20 hours. The culture solution was drawn into 25 mL of YPG medium at an inoculation amount of 1%, cultured at 230 rpm and 30° C. for 18-20 h until OD600=1.0; then the culture solution was centrifuged at 3000 rpm for 5 min to collect the bacteria, and the bacteria were resuspended with 25 ml of YPM, placed in a 250 mL Erlenmeyer flask, and cultured at 230 rpm, 30° C. for 72 h; during the culture period, methanol with a final concentration of 1% was added every 24 h to induce expression.
5. Purification of protein: the fermentation broth was centrifuged and the supernatant was collected; the affinity chromatography column was equilibrated with equilibrium solution, and the recombinant protein carrying the His-tag was separated and purified by nickel column affinity chromatography. SDS-PAGE (FIG. 3, panels D, E and F) and Western blotting (FIG. 3, panels d, e and f) were performed for the molecular weight and immunological verification of the recombinant proteins (wherein, the antibody was 6×-His tag polyclonal antibody, purchased from Thermo Fisher). The purified protein samples were desalted through a G25 column to obtain a high-purity fusion protein.

In summary, the recombinant expression vectors pPICZαA-rhCol-rhFN, pPICZαA-rhCol and pPICZαA-rhFN were obtained by cloning the human collagen-human fibronectin gene, human collagen gene, and human fibronectin gene into Pichia pastoris expression vector pPICZαA through gene cloning technology. Yeast competent cells were transfected by electroporation to obtain recombinant expression strains. The expression of recombinant strains was induced by methanol, and the purified fusion protein was obtained by nickel affinity chromatography, and the molecular weight and immunological verification of the recombinant protein were verified by SDS-PAGE and Western blot.

The invention provides a method for highly expressing human collagen-human fibronectin rhCol-rhFN fusion protein, human collagen rhCol, and human fibronectin rhFN in Pichia pastoris, and the biological activities thereof were detected. The method comprises the following steps:

Cell adhesion assay: HF-MSC cells (ATCC No: CM-1252) were cultured in DMEM/F12 containing 10% FBS, at 37° C., CO₂ 5%; washed with PBS for once, then added with 0.25% Trypsin-EDTA trypsin solution for digestion, centrifuged to collect cells; resuspended with DMEM/F12 with a cell density at 6×10⁴ cells/mL, and the cell suspension (cell density at 1.5×10⁴ cells/mL) was respectively inoculated into a low-adhesion 96-well plate coated with rhCol-rhFN protein, rhCol protein, and rhFN protein (each protein concentration was 1 μmol/L) at the bottom. The cells were cultured at 37° C. for 5 hours, with CO₂ concentration maintained at 5%; non-adherent cells were washed off with PBS; the cells were counted by a phase contrast microscope and the numbers of cells in each group were compared with the MTT method. PBS solution was used as a negative control. The results showed that the fusion protein rhCol-rhFN, recombinant rhCol protein, and recombinant rhFN protein provided by the present invention could all promote HF-MSC (ATCC No: CM-1252) cell adhesion, and the adhered cells were in good growth state. The fusion protein rhCol-rhFN was observed under a microscope not only to promote cell adhesion, but also to significantly promote cell extension. The cell adhesion performance was significantly higher than that of recombinant protein rhCol and recombinant protein rhFN. The results were shown in FIG. 4.

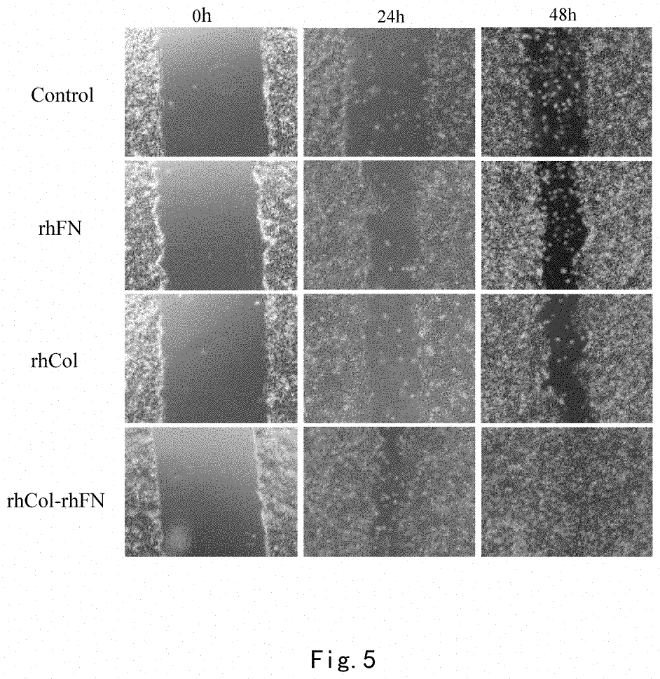

Detection of cell wound-healing ability: HF-MSC cells were digested with trypsin and inoculated into a 12-well plate. Regarding the number of cells, it was advisable to cover the bottom of the plate after cell adhesion. After the bottom of the plate was covered by cells, a 100 μl pipette tip was used to be perpendicular to the plate and make cell scratches along the same position as the line on the back of the plate, and the width of each scratch should be as same as possible. The cell culture medium was aspirated, the plate was washed three times with PBS, and cell debris produced by the scratch was washed away. The medium (serum concentration: 1%) containing rhCol-rhFN fusion protein, human collagen rhCol, and human fibronectin rhFN (both at a concentration of 1 μmol/L) was added and then photographed for recording. The culture plate was placed into an incubator and cultured for 12 h and 24 h, and then photographed respectively. The results were shown in FIG. 5. The healing rates of rhCol-rhFN group were significantly different at 12 h and 24 h.

In summary, human fibronectin rhFN, human collagen rhCol and human collagen-human fibronectin fusion protein rhCol-rhFN were expressed respectively through genetic engineering in the present invention. The results of in vitro cell experiments showed that the fused human collagen-human fibronectin rhCol-rhFN had obvious effects on cell adhesion promotion and wound repair, and the effects were significantly better than those of human fibronectin rhFN and human collagen rhCol expressed alone.

The present invention provides a method for highly expressing a fusion protein rhCol-rhFN of human collagen and human fibronectin in Pichia pastoris, and the skin allergy thereof was detected. The method comprises the following steps:

32 KM mice (purchased from Guangdong Medical Laboratory Animal Center, license number: SCXK (Guangdong) 2019-0035) were selected (18-22 g, half male and half female). On the first day, the animals were grouped, labeled, weighed, and recorded for clinical symptoms. 1% sodium lauryl sulfate (SLS) was coated evenly to the skin on the back of both ears of the mice. The brush or cotton swab should be soaked in SDS solution and smeared repeatedly 4-5 times on the back of each ear of the mouse. 1 hour later, rhCol-rhFN fusion protein, rhCol, rhFN, small molecular animal-derived collagen (purchased from Shandong Longbei Biotechnology Co., Ltd., catalog number SC13137078101884), large molecular animal-derived collagen (purchased from Xiya Chemical Technology (Shandong) Co., Ltd., catalog number A15847) or positive control (0.01% SLS solution) were smeared at 25 μL/ear respectively. The operations on day 2, day 3, and day 7 were the same as that on day 1, and the pretreatment by 0.01% SLS solution and the smearing of rhCol-rhFN fusion protein, rhCol, rhFN, small molecular animal-derived collagen, and large molecular animal-derived collagen were repeated. No treatment was done on day 4 to day 6. On day 8, the body weight and any clinical symptoms of the mice were recorded. About 24-30 hours after smearing of 0.01% SLS solution and rhCol-rhFN fusion protein, rhCol, rhFN, small molecular animal-derived collagen, and large molecular animal-derived collagen on day 7, the animals were humanely sacrificed, the bilateral submandibular lymph nodes of the mice were removed, and the auricles were separated and punched (diameter: 6 mm) and weighed. The removed lymph nodes were cut up in a lysis buffer and lysed on ice. After the lysis was completed, the lymph nodes were centrifuged and supernatant was obtained as a sample to be tested; the sample to be tested was analyzed by chemiluminescence to obtain CPS values (the number of fluorescent photons received per second). ATP assay kit (purchased from Beyotime Biotech Co., Ltd.) was used to measure the ATP content in lymph nodes, and the bioluminescence was expressed as relative luminescence units (RLU). The period from sacrificing to measuring the ATP content of each animal was consistent, i.e., within about 30 minutes. The series of operations from submandibular lymph node separation to ATP assay must be completed within 20 minutes for each animal, and the treatment time for each animal should be consistent.

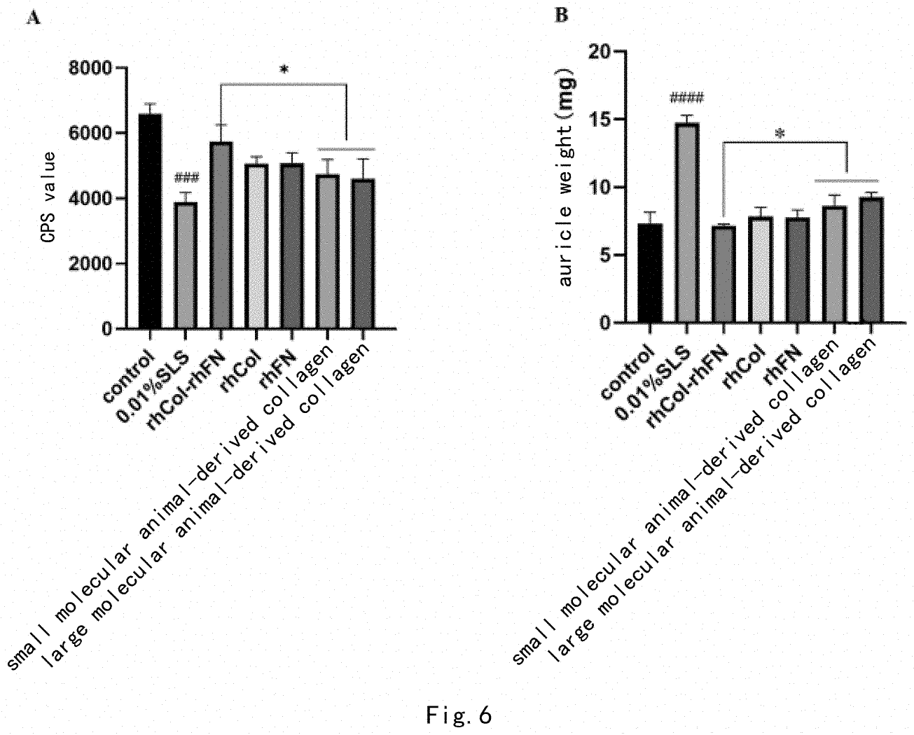

The results were shown in FIG. 6. No death or abnormality occurred in the tested mice during the observation period, and the recombinant human collagen-human fibronectin rhCol-rhFN fusion protein had lower sensitization ability than animal-derived collagens.

In summary, human fibronectin rhFN, human collagen rhCol and human collagen-human fibronectin fusion protein rhCol-rhFN were expressed respectively through genetic engineering in the present disclosure. The results of local lymph node tests in mice showed that the fused human collagen-human fibronectin rhCol-rhFN had a lower sensitization ability, and the sensitization ability was significantly lower than that of animal-derived collagen.

It should be understood by those skilled in the art that although the present invention has been specifically described with reference to the above-mentioned Examples, the present invention is not limited to these specific Examples. Based on the methods and technical solutions taught in the present invention, without departing from the spirit of the present invention, those skilled in the art can make appropriate modifications or improvements, and the resulting equivalent embodiments are all within the scope of the present invention.

1. A fusion protein, which is obtained by fusion of human collagen and human fibronectin.

2. The fusion protein according to claim 1, wherein the fusion protein comprises at least one domain of human type I collagen alpha chain and at least one domain of human type III fibronectin.

3. The fusion protein according to claim 1, wherein the human collagen (rhCol) of the fusion protein is a short peptide formed by repeats of 10 to 50 amino acids, the number of repeats is 10 to 20, and the amino acid sequence is SEQ ID NO.1, SEQ ID NO.2, or SEQ ID NO.3.

4. The fusion protein according to claim 2, wherein the human type III fibronectin (rhFN) of the fusion protein is one or more domains in 8-15 domains, and the amino acid sequence is SEQ ID NO.4, SEQ ID NO.5, or SEQ ID NO.6.

5. The fusion protein according to claim 1, wherein the N or C terminus of the human collagen is fused with the N or C terminus of the human fibronectin.

6. The fusion protein according to claim 1, wherein the human collagen is fused with the human fibronectin by a linker peptide; the linker peptide is preferably (GGGS)n, wherein n is an integer of 1-5, preferably, the fusion protein further includes a Kex2 protease sequence, a secretion signal peptide sequence, a His tag for protein purification, or a combination thereof.

7. The fusion protein according to claim 1, wherein the amino acid sequence of the fusion protein is SEQ ID NO.7, SEQ ID NO.8, or SEQ ID NO.9.

8. A nucleic acid encoding the fusion protein according to claim 1.

9. An expression vector comprising the nucleic acid according to claim 8, preferably the expression vector is selected from pPICZαA, pHIL, pPIC9k, and/or pPICZαB, more preferably pPICZαA or pHIL, most preferably pPICZαA.

10. A host cell comprising the expression vector according to claim 9, preferably the host cell is a yeast cell, more preferably a Pichia cell, still more preferably GS115, X33 and KM71, most preferably GS115.
